(19) United States
(12) Patent Application Publication
(10) Pub. No.: US 2024/0342157 A1
(43) Pub. Date: Oct. 17, 2024

(54) BAK ACTIVATORS, PHARMACEUTICAL COMPOSITIONS, AND USES IN TREATING CANCER

(71) Applicant: Emory University, Atlanta, GA (US)

(72) Inventor: Xingming DENG, Atlanta, GA (US)

(21) Appl. No.: 18/580,674

(22) PCT Filed: Jul. 21, 2022
(86) PCT No.: PCT/US2022/037783

§ 371 Date: Jan. 19, 2024

Related U.S. Application Data
(60) Provisional application No. 63/224,112, filed on Jul. 21, 2021.

(51) Int. Cl. A61K 31/473; A61K 45/06; A61P 35/00

(52) U.S. Cl. CPC A61K 31/473; A61K 45/06; A61P 35/00

(57) ABSTRACT

This disclosure relates to activators of Bak, pharmaceutical compositions, and uses in treating cancer. In certain embodiments, this disclosure relates to methods of treating cancer comprising administering an effective amount of a Bak activator as disclosed herein to a human subject in need thereof. In certain embodiments, this disclosure relates to pharmaceutical compositions comprising a Bak activator which is 1-((2-((2-methoxyacridin-9-yl)amino)ethyl)amino)propan-2-ol (BKA-073), derivative, ester, or salt thereof and a pharmaceutically acceptable excipient.

### CROSS-REFERENCE TO RELATED APPLICATIONS

This application claims the benefit of U.S. Provisional Application No. 63/224,112 filed Jul. 21, 2021. The entirety of this application is hereby incorporated by reference for all purposes.

### STATEMENT REGARDING FEDERALLY SPONSORED RESEARCH OR DEVELOPMENT

This invention was made with government support under CA200905 awarded by the National Institutes of Health. The government has certain rights in the invention.

### BACKGROUND

Lung cancer is often classified as non-small cell lung cancer or small cell lung cancer. Non-small cell lung cancer accounts for vast majority of lung cancers. The standard of care for advanced small cell lung cancer and non-small cell lung cancer includes radiation and chemotherapy. Lung cancer is a global health problem. For example, in the United States, more patients die from lung cancer alone than prostate, breast, and colon cancers combined. Thus, there is a need to identify improved therapies.

Iyer et al. report robust autoactivation for apoptosis by BAK but not BAX highlights BAK as an important therapeutic target. Cell Death and Disease, 2020, 11:268.

Kalirajan et al. report oxazine substituted 9-anilinoacridine derivatives and evaluation for their antioxidant and anticancer activities. European Journal of Medicinal Chemistry, 2012, 56 217-224.

Gellerman et al. report 9-aminoacridine derivatives as potential candidates for cancer treatment. WO 2011/0519550.

Park et al. report discovery of small molecule Bak activator for lung cancer therapy. Theranostics, 2021, 11(17): 8500-8516.

References cited herein are not an admission of prior art.

### SUMMARY

This disclosure relates to activators of Bak, pharmaceutical compositions, and uses in treating cancer. In certain embodiments, this disclosure relates to methods of treating cancer comprising administering an effective amount of a Bak activator which is 1-((2-((2-methoxyacridin-9-yl)amino)ethyl)amino)propan-2-ol (BKA-073), derivative, prodrug, ester, or salt thereof. In certain embodiments, this disclosure relates to methods of treating cancer comprising administering an effective amount of a Bak activator as disclosed herein to a human subject in need thereof optionally in combination with other chemotherapy agents or therapeutic methods.

In certain embodiments, the subject is a human patient.

In certain embodiments, the cancer is a metastatic cancer, solid cancer, or hematological cancer. In certain embodiments, the subject is diagnosed with lung cancer, small cell lung cancer, or non-small cell lung cancer (NSCLC). In certain embodiments, the subject is diagnosed with a cancer selected from breast cancer, colon cancer, lymphoma, multiple myeloma, pancreatic cancer (PANC-1) and osteosarcoma.

In certain embodiments, the activator of Bak as disclosed herein is administered in combination with an additional chemotherapy agent. In certain embodiments, the chemotherapy agent is a Bcl-2 inhibitor such as venetoclax, navitoclax, obatoclax, or sabutoclax. In certain embodiments, the chemotherapy agent is cisplatin, carboplatin, paclitaxel, albumin-bound paclitaxel, docetaxel, gemcitabine, vinorelbine, etoposide, pemetrexed, or combinations thereof.

In certain embodiments, the chemotherapy agent is a combination of cisplatin or carboplatin plus etoposide, paclitaxel, or gemcitabine with vinorelbine.

In certain embodiments, this disclosure relates to methods of diagnosing and treating a subject with cancer comprising measuring levels of Bak from a sample of the subject; comparing the measured levels of Bak to a reference or normal value; wherein if the measured levels are higher than the reference or normal values, administering an effective amount of a Bak activator, alternative chemotherapy treatment, a combination chemotherapy treatment, or an aggressive chemotherapy treatment to the subject.

In certain embodiments, the subject is diagnosed with a cancer causing KRAS-mutation, e.g., KRAS (G12C, G12D and G12R).

In certain embodiments, the disclosure relates to the production of a medicament comprising a Bak activator as disclosed herein, e.g., 1-((2-((2-methoxyacridin-9-yl)amino)ethyl)amino)propan-2-ol, derivative, ester, prodrug, or salt thereof for use in the treatment of cancer.

In certain embodiments, this disclosure relates to pharmaceutical compositions comprising an activator of Bak as disclosed herein or pharmaceutically acceptable salt thereof and a pharmaceutically acceptable excipient. In certain embodiments, the pharmaceutical is in the form of a pill, capsule, or table. In certain embodiments, the pharmaceutical composition is in the form of an aqueous isotonic or non-isotonic pH buffered solution.

### BRIEF DESCRIPTION OF THE SEVERAL VIEWS OF THE DRAWINGS

FIG. 1A illustrates the chemical structure of the Bak activator-073 (BKA-073) with the chemical name 1-((2-((2-methoxyacridin-9-yl)amino)ethyl)amino)propan-2-ol.

FIG. 1B shows data indicating BKA-073 is compound that targets BH3 domain of Bak and induces mitochondrial priming and apoptosis in lung cancer cells. Expression levels of Bak were analyzed by Western blot in NSCLC and SCLC cell lines. A panel of NSCLC and SCLC cell lines were treated with BKA-073 (1 μM) for 16 h or 72 h, followed by analysis of dynamic BH3 profiling or apoptotic cell death.

FIG. 1C shows data indicating BKA-073 induces mitochondrial priming and apoptosis. Expression levels of Bak were analyzed by Western blot in various type of cancer cell lines. Cancer cell lines were treated with BKA-073 (1 μM) for 16 h or 72 h, followed by analysis of dynamic BH3 profiling or apoptotic cell death.

FIGS. 2A-2B shows data indicating BKA-073 specifically binds to Bak inducing Bak oligomerization.

FIG. 2A shows data from fluorescence polarization assays that were performed to measure the inhibitory constant (Ki). Purified Bak protein or other Bcl2 family member(s), BKA-073, and fluorescence-labeled Bak BH3 peptide were used.

FIG. 2B shows data on the binding affinity of BKA-073 with WT Bak or delta BH3 Bak deletion mutant protein which was examined by isothermal titration calorimetry assays. The binding constant (KD) value was determined by fitting of the titration curve to a 1-site binding mode.

FIG. 3 shows data indicating BKA-073 potently suppresses lung cancer growth in a dose-dependent manner in vivo. Nu/Nu mice bearing A549 lung cancer xenografts were treated with increasing doses of BKA-073 (5 to 15 mg/kg/d) i.p. for 28 days. Tumor volume was measured once every 2 days. After treatment, mice were sacrificed, and tumors were removed and analyzed.

FIG. 4 shows data indicating BKA-073 suppresses SCLC in xenografts and PDX models. Nu/Nu mice bearing xenografts derived from SCLC cell line DMS114 or from SCLC patients (TKO-2 or TKO-5) and were treated with BKA-073 (15 mg/kg/d) i.p. for 14 or 28 days. Tumor volume was measured once every 2 days. After treatment, mice were sacrificed, and tumors were removed and analyzed.

FIG. 5 shows data indicating BKA-073 suppresses prolongs survival in genetically engineered mouse models (GEMMs). After administration of adenovirus Cre recombinase in KRAS G12D LKB1fl/fl (KL) mice for 6 weeks, KL mice were treated with KRA-073 (15 mg/kg/d) i.p. for 48 days (n=6 mice/group). Survival of mice is shown for up to 48 days before euthanization in the control group versus the BKA-073 treatment group.

FIG. 6 shows data indicating BKA-073 synergizes with the Bcl-2 inhibitor ABT-199 (venetoclax) against SCLC and NSCLC in vitro and in vivo. Nu/Nu mice carrying SCLC DMS53 xenografts or NSCLC H460 xenografts were treated with BKA-073 (10 mg/kg/d) i.p., ABT-199 (60 mg/kg/d) orally, or the combination for 28 days. Tumor volume was measured once every 2 days. After treatment, mice were sacrificed, and tumors were removed and analyzed.

FIG. 7 shows data indicating high levels of Bak expression are associated with poor prognosis in NSCLC patients. Kaplan-Meier survival curve of NSCLC patients, n=208.

### DETAILED DISCUSSION

Before the present disclosure is described in greater detail, it is to be understood that this disclosure is not limited to embodiments described, and as such may, of course, vary. It is also to be understood that the terminology used herein is for the purpose of describing particular embodiments only and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

An “embodiment” refers to an example and is not necessarily limited to such example. Embodiments of the present disclosure will employ, unless otherwise indicated, techniques of medicine, organic chemistry, biochemistry, molecular biology, pharmacology, and the like, which are within the skill of the art. Such techniques are explained fully in the literature.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

To the extent that any chemical formulas reported herein contain one or more chiral centers, the formulas are intended to encompass all stable stereoisomers, enantiomers, and diastereomers. It is also understood that formulas encompass all tautomeric forms.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Bak,” also referred to as “Bcl-2 homologous antagonist/killer,” is a pore-forming pro-apoptotic protein containing a BH3 domain; thus, categorized as a BCL-2 family protein. BCL2 family members form oligomers or heterodimers and act as regulators for variety of cellular activities Bak is reported to activate apoptosis within the mitochondria. Human [Homo sapiens] Bcl-2 homologous antagonist/killer is denoted as NCBI Reference Sequence: NP_001179.1.

As used herein, “subject” refers any animal, preferably a human patient, livestock, or domestic pet.

As used herein, the terms “treat” and “treating” are not limited to the case where the subject (e.g., human patient) is cured and the disease is eradicated. Rather, embodiments, of the present disclosure also contemplate treatment that merely reduces symptoms, and/or delays disease progression.

As used herein, the term “combination with” when used to describe administration with an additional treatment means that the agent may be administered prior to, together with, or after the additional treatment, or a combination thereof.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In certain embodiments, the salts are conventional nontoxic pharmaceutically acceptable salts including the quaternary ammonium salts of the parent compound formed, and non-toxic inorganic or organic acids.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom, or replacing an amino group with a hydroxyl group. The derivative may be a prodrug. Derivatives may be prepared by any variety of synthetic methods or appropriate adaptations presented in synthetic or organic chemistry textbooks, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O) OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, or heteroarylalkyl.

As used herein, the term “prodrug” refers a compound that, after administration, is metabolized (i.e., converted within the body) into a pharmacologically active drug. Examples include alkoxy esters of hydroxyl groups or carboxyl groups such as acetate esters, benzoate esters, alkyl ethers, amino acids esters, glycolic acid esters, malic acid esters, acyloxyalkyl esters, alkoxycarbonyloxy alkyl esters, S-acylthioalkyl esters, hydroxylamine amides, phosphonylmethoxy ethers, phosphates, phosphoramidates, and combinations thereof.

The prodrug may also have improved solubility in pharmaceutical compositions over the parent drug. A prodrug may be converted into the parent drug by various mechanisms, including enzymatic processes and metabolic hydrolysis. Typical prodrugs are pharmaceutically acceptable esters. Prodrugs include compounds wherein a hydroxy, amino or mercapto group is bonded to any group that, when the prodrug of the active compound is administered to a subject, cleaves to form a free hydroxy, free amino or free mercapto group, respectively.

If a disclosed compound or a pharmaceutically acceptable form of the compound contains an alcohol functional group, a prodrug can be formed by the replacement of the hydrogen atom of the alcohol group with a group such as (C₁-C₆)(alkanoyloxy)methyl, 1-((C₁-C₆)alkanoyloxy) ethyl, 1-methyl-1((C₁-C₆)alkanoyloxy)ethyl (C₁-C₆)(alkoxycarbonyloxy)methyl, N—(C₁-C₆)alkoxycarbonylaminomethyl, succinoyl, (C₁-C₆)alkanoyl, alpha-amino(C₁-C₄)alkanoyl, arylacyl and alpha-aminoacyl, or alpha-aminoacyl-alpha-aminoacyl, where each alpha-aminoacyl group is independently selected from naturally occurring L-amino acids —P(O)(OH)₂, —P(O)(O(C₁-C₆)alkyl)₂, and glycosyl (the radical resulting from the removal of a hydroxyl group of the hemiacetal form of a carbohydrate).

If a disclosed compound or a pharmaceutically acceptable form of the compound incorporates an amine functional group, a prodrug can be formed by the replacement of a hydrogen atom in the amine group with a group such as R-carbonyl, RO-carbonyl, NRR′-carbonyl where R and R′ are each independently (C₁-C₁₀)alkyl, (C₃-C₇)cycloalkyl, benzyl, a natural alpha-aminoacyl, —C(OH)C(O)OY₁ wherein Y¹ is H, (C₁-C₆)alkyl or benzyl, —C(OY₂)Y₃ wherein Y₂ is (C₁-C₄) alkyl and Y₃ is (C₁-C₆)alkyl, carboxy(C₁-C₆)alkyl, amino(C₁-C₄)alkyl or mono-N or di-N,N—(C₁-C₆)alkylaminoalkyl, —C(Y₄)Y₅ wherein Y₄ is H or methyl and Y₅ is mono-N— or di-N,N—(C₁-C₆)alkylamino, morpholino, piperidin-1-yl or pyrrolidin-1-yl.

As used herein, “alkyl” means a noncyclic straight chain or branched, unsaturated or saturated hydrocarbon such as those containing from 1 to 25 carbon atoms. For example, a “C₈-C₁₈” refers to an alkyl containing 8 to 18 carbon atoms. Likewise, a “C₆-C₂₂” refers to an alkyl containing 6 to 22 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, and the like.

The term “aryl” refers to aromatic homocyclic (i.e., hydrocarbon) mono-, bi- or tricyclic ring-containing groups preferably having 6 to 12 members such as phenyl, naphthyl and biphenyl. Phenyl is a preferred aryl group.

As used herein, “heteroaryl” or “heteroaromatic” refers an aromatic heterocarbocycle having 1 to 4 heteroatoms selected from nitrogen, oxygen, and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isoxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen, and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkoxy” refers to an alkyl group as defined above with the indicated number of carbon atoms attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkoxyalkyl” refers an alkyl group as defined above with the indicated number of carbon atoms attached through an alkyl bridge (i.e., —CH₂—O—CH₂CH₃).

“Alkylamino” refers an alkyl group as defined above with the indicated number of carbon atoms attached through an amino bridge. An example of an alkylamino is methylamino, (i.e., —NH—CH3).

“Alkylthio” refers to an alkyl group as defined above with the indicated number of carbon atoms attached through a sulfur bridge. An example of an alkylthio is methylthio, (i.e., —S—CH₃).

“Alkanoyl” refers to an alkyl as defined above with the indicated number of carbon atoms attached through a carbonyl bride (i.e., —(C═O)alkyl).

The terms “cycloalkyl” and “cycloalkenyl” refer to mono-, bi-, or tri homocyclic ring groups of 3 to 15 carbon atoms which are, respectively, fully saturated, and partially unsaturated.

“Alkylsulfonyl” refers to an alkyl as defined above with the indicated number of carbon atoms attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above with the indicated number of carbon atoms attached through a sulfamoyl bridge (i.e., —NHS(═O)₂alkyl), and an “Arylsulfamoyl” refers to an alkyl attached through a sulfamoyl bridge (i.e., (i.e., —NHS(═O)₂aryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

In certain embodiments, this disclosure contemplates compound or composition as disclosed herein in the production of a medicament for use in treating cancer. “Cancer” refers any of various cellular diseases with malignant neoplasms characterized by the proliferation of cells. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others.

The cancer to be treated in the context of the present disclosure may be any type of cancer or tumor such as lung cancer, non-small cell lung cancer and subtypes of NSCLC such as adenocarcinoma, squamous cell carcinoma, and large cell carcinoma, and small cell lung cancer. Contemplated are malignancies located in the colon, abdomen, bone, breast, digestive system, liver, pancreas, peritoneum, endocrine glands (adrenal, parathyroid, hypophysis, testicles, ovaries, thymus, thyroid), eye, head and neck, nervous system (central and peripheral), lymphatic system, pelvis, skin, soft tissue, spleen, thorax and genito-urinary apparatus and, more particularly, adrenocortical carcinoma, AIDS-related lymphoma, AIDS-related malignant tumors, anal cancer, astrocytoma, cancer of the biliary tract, cancer of the bladder, bone cancer, brain stem glioma, brain tumors, breast cancer, cancer of the renal pelvis and ureter, primary central nervous system cerebellar astrocytoma, brain astrocytoma, cancer of the cervix, chronic lymphocytic leukemia, chronic myeloid leukemia, cancer of the colon, cutaneous T-cell lymphoma, endocrine pancreatic islet cells carcinoma, endometrial cancer, ependymoma, epithelial cancer, cancer of the esophagus, Ewing's sarcoma and related tumors, cancer of the exocrine pancreas, extracranial germ cell tumor, extragonadal germ cell tumor, extrahepatic biliary tract cancer, cancer of the eye, Gaucher's disease, cancer of the gallbladder, gastric cancer, gastrointestinal carcinoid tumor, gastrointestinal tumors, germ cell tumors, gestational trophoblastic tumor, head and neck cancer, hepatocellular cancer, hypergammaglobulinemia, hypopharyngeal cancer, Hodgkin's disease, intestinal cancers, intraocular melanoma, islet cell carcinoma, islet cell pancreatic cancer, Kaposi's sarcoma, cancer of the larynx, cancer of the lip and mouth, macroglobulinemia, malignant mesothelioma, malignant thymoma, medulloblastoma, melanoma, mesothelioma, occult primary metastatic squamous neck cancer, primary metastatic squamous neck cancer, metastatic squamous neck cancer, multiple myeloma, multiple myeloma/plasmatic cell neoplasia, myelodysplastic syndrome, myelogenous leukemia, myeloid leukemia, myeloproliferative disorders, paranasal sinus and nasal cavity cancer, nasopharyngeal cancer, neuroblastoma, non-Hodgkin's lymphoma, non-melanoma skin cancer, non-small cell lung cancer, metastatic squamous neck cancer with occult primary, buccopharyngeal cancer, malignant fibrous histiocytoma, malignant fibrous osteosarcoma/histiocytoma of the bone, epithelial ovarian cancer, ovarian germ cell tumor, ovarian low malignant potential tumor, pancreatic cancer, paraproteinemias, purpura, parathyroid cancer, cancer of the penis, hypophysis tumor, neoplasia of plasmatic cells/multiple myeloma, primary central nervous system lymphoma, primary liver cancer, prostate cancer, rectal cancer, renal cell cancer, cancer of the renal pelvis and ureter, retinoblastoma, rhabdomyosarcoma, cancer of the salivary glands, sarcoidosis, sarcomas, skin cancer, small cell lung cancer, small intestine cancer, soft tissue sarcoma, squamous neck cancer, stomach cancer, pineal and supratentorial primitive neuroectodermal tumors, T-cell lymphoma, testicular cancer, thymoma, thyroid cancer, transitional cell cancer of the renal pelvis and ureter, transitional renal pelvis and ureter cancer, trophoblastic tumors, cell cancer of the renal pelvis and ureter, cancer of the urethra, cancer of the uterus, uterine sarcoma, vaginal cancer, optic pathway and hypothalamic glioma, cancer of the vulva, Waldenstrom's macroglobulinemia, Wilms' tumor and any other hyperproliferative disease, as well as neoplasia, located in the system of a previously mentioned organ.

In certain embodiments, compounds disclosed herein may be administered in combination with an additional anti-cancer agent. A “chemotherapy agent,” “chemotherapeutic,” “anti-cancer agent” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); adriamycin, bleomycin, vinblastine, dacarbazine (ABVD); cyclophosphamide, doxorubicin, vincristine, prednisolone (CHOP); rituximab, cyclophosphamide, doxorubicin, vincristine, prednisolone (RCHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC). In certain embodiments, the chemotherapy agent is an antibody, anti-PD-1, anti-PD-L1, anti-CTLA4 antibody or combinations thereof, such as an anti-CTLA4 (e.g., ipilimumab, tremelimumab), an anti-PC-L1 (e.g., atezolizumab, avelumab, durvalumab) or an anti-PD1 antibody (e.g., nivolumab, pembrolizumab, cemiplimab, dostarlimab, spartalizumab, camrelizumab, tislelizumab, toripalimab, sintilimab).

### Bak Activators

Although it is not intended that certain embodiments, of this disclosure be limited by any particular mechanism, it is believed that certain compounds disclosed herein active Bak; thus, the compounds are useful as therapeutic agents for treating cancer.

In certain embodiments, the Bak activator is 1-((2-((2-methoxyacridin-9-yl)amino)ethyl)amino)propan-2-ol (BKA-073), derivative, prodrug, ester, or salt thereof. In certain embodiments, the derivative is a compound of formula I or II,

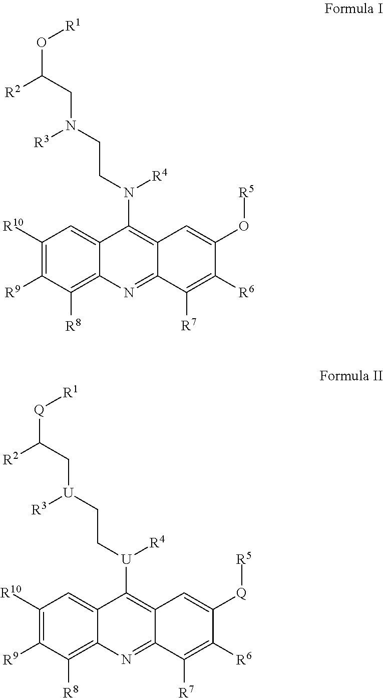

derivative, prodrug, ester, or salts thereof, wherein:
Q is O or S;
U is N or CH;
R¹, R², R³, R⁴, R⁵, R⁶, R⁷, R⁸, R⁹, and R¹⁰ are each individually and independently hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, phosphate, alkylsulfinyl, alkylsulfonyl, arylsulfonyl, carbocyclyl, aryl, or heterocyclyl, wherein R¹, R², R³, R⁴, R⁵, R⁶, R⁷, R⁸, R⁹, and R¹⁰ are optionally substituted with one or more, the same or different, R¹¹;
R¹¹ is alkyl, halogen, cyano, hydroxy, amino, mercapto, formyl, carboxy, carbamoyl, alkoxy, alkanoyl, alkylthio, alkylamino, phosphate, aminoalkyl, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹²;
R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R¹ is hydrogen. In certain embodiments, R² is alkyl. In certain embodiments, R³ is hydrogen. In certain embodiments, R⁴ is hydrogen. In certain embodiments, R⁵ is alkyl or methyl. In certain embodiments, R⁶, R⁷, R⁸, R⁹, and R¹⁰ are hydrogen. In certain embodiments, Q is O. In certain embodiments, U is NH.

### Pharmaceutical Compositions

In certain embodiments, this disclosure relates to pharmaceutical compositions comprising Bak activators disclose herein and a pharmaceutically acceptable excipient. In certain embodiments, the pharmaceutically acceptable excipient is selected from a diluent, disintegrant, solubilizing agent, or a lubricant.

In certain embodiments, the pharmaceutically acceptable excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, maltitol, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, methyl paraben, propyl paraben, and combinations thereof.

In certain embodiments, the pharmaceutically acceptable excipient is a diluent. Examples include microcrystalline cellulose, other diluents may be, for example: calcium carbonate, calcium phosphate, calcium sulfate, cellulose acetate, erythritol, ethylcellulose, fructose, inulin, isomalt, lactitol, lactose, magnesium carbonate, magnesium oxide, maltitol, maltodextrin, maltose, mannitol, polydextrose, polyethylene glycol, pullulan, simethicone, sodium bicarbonate, sodium carbonate, sodium chloride, sorbitol, starch, sucrose, trehalose, and xylitol.

In certain embodiments, the pharmaceutically acceptable excipient is a disintegrant. Examples of a disintegrant may be, for example: alginic acid, calcium alginate, carboxymethylcellulose calcium, chitosan, colloidal silicon dioxide, croscarmellose sodium, crospovidone, glycine, guar gum, hydroxypropyl cellulose, low-substituted hydroxypropyl cellulose, magnesium aluminum silicate, methylcellulose, povidone, sodium alginate, sodium carboxymethylcellulose, sodium starch glycolate and starch.

In certain embodiments, the pharmaceutically acceptable excipient is a solubilizing agent. Examples of a solubilizing agent may be, for example: benzalkonium chloride, benzyl benzoate, sulfobutyl ether β-cyclodextrin sodium, cetylpyridinium chloride, cyclodextrins, diethylene glycol monoethyl ether, fumaric acid, hydroxypropyl beta cyclodextrin, hypromellose, lanolin alcohols, lecithin, oleyl alcohol, phospholipids, poloxamer, polyoxyethylene alkyl ethers, polyoxyethylene castor oil derivatives, polyoxyethylene sorbitan fatty acid esters, polyoxyl hydroxystearate, polyoxylglycerides, povidone, pyrrolidone, sodium lauryl sulfate, sorbitan esters (sorbitan fatty acid esters), tricaprylin, triolein and vitamin E polyethylene glycol succinate.

In certain embodiments, the pharmaceutically acceptable excipient is a lubricant. Examples of a lubricant may be, for example calcium stearate, glyceryl behenate, glyceryl dibehenate, glyceryl monostearate, glyceryl palmitostearate, a mixture of behenate esters of glycerine (e.g. a mixture of glyceryl dibehenate, tribehenin and glyceryl behenate), leucine, magnesium stearate, myristic acid, palmitic acid, poloxamer, polyethylene glycol, potassium benzoate, sodium benzoate, sodium lauryl sulfate, sodium stearate, sodium stearyl fumarate, stearic acid, talc, tribehenin and zinc stearate.

In certain embodiments, the pharmaceutically acceptable excipient is selected from lactose, sucrose, mannitol, triethyl citrate, dextrose, cellulose, methyl cellulose, ethyl cellulose, hydroxyl propyl cellulose, hydroxypropyl methylcellulose, carboxymethylcellulose, croscarmellose sodium, polyvinyl N-pyrrolidone, crospovidone, ethyl cellulose, povidone, methyl and ethyl acrylate copolymer, polyethylene glycol, fatty acid esters of sorbitol, lauryl sulfate, gelatin, glycerin, glyceryl monooleate, silicon dioxide, titanium dioxide, talc, corn starch, carnauba wax, stearic acid, sorbic acid, magnesium stearate, calcium stearate, castor oil, mineral oil, calcium phosphate, starch, carboxymethyl ether of starch, iron oxide, triacetin, acacia gum, esters, or salts thereof.

In certain embodiments, the pharmaceutical composition is in the form of a tablet, pill, capsule, gel, gel capsule or cream. In certain embodiments, the pharmaceutical composition is in the form of a sterilized pH buffered aqueous salt solution or a saline phosphate buffer between a pH of 6 to 8, optionally comprising a saccharide or polysaccharide.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response, and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange.

Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

Pharmaceutically acceptable salts derived from appropriate bases include alkali metal, alkaline earth metal, ammonium, or quaternary ammonium, e.g., N⁺(C₁₋₄alkyl)₄, salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate. Organic bases from which salts can be derived include, for example, primary, secondary, and tertiary amines, substituted amines including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, a Bak activator disclosed herein may be used in the “free base form” or as a pharmaceutically acceptable salt, or as any mixture thereof. In one embodiment the Bak activator is in the free base form. It is understood that “free base form” refers to the case where the Bak activator is not in the form of a salt.

In certain embodiments, this disclosure relates to kits or pharmaceutical packaging comprising a Bak activator or combinations of agents disclosed herein with instructions for use. In certain embodiments, the individual agent may be packaged in a container, e.g., vial, box, syringe, or bottle. In certain embodiments, instructions may be in a pamphlet inside a container or on the outside or inside of the container.

### Methods of Use

In certain embodiments, this disclosure relates to methods of treating cancer comprising administering an effective amount of a Bak activator or pharmaceutical composition containing the same to a subject in need thereof. In certain embodiments, the subject is a human patient. In certain embodiments, the Bak activator induces or increases cellular apoptose, e.g., formation of Bak oligomers in mitochondria promotes cytochrome c (Cyt c) release to induce apoptosis. In certain embodiments, this disclosure relates to the production of a medicament comprising a Bak activator disclosed herein for use in treating cancer.

In certain embodiments, the cancer is a metastatic cancer, solid cancer, or hematological cancer. In certain embodiments, the subject is diagnosed with lung cancer, small cell lung cancer, or non-small cell lung cancer (NSCLC). In certain embodiments, the subject is diagnosed with a cancer selected from breast cancer, colon cancer, lymphoma, multiple myeloma, pancreatic cancer (PANC-1), and osteosarcoma.

In certain embodiments, the subject is diagnosed with a cancer selected from lung, pancreatic, colorectal, uterine, esophageal, gastric, cervical, breast, prostate, or bladder cancer. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the Bak activator is administered in combination with an additional chemotherapy agent. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with non-small cell lung cancer (NSCLC). In certain embodiments, malignant cells are seen on sputum cytology. In certain embodiments, a tumor can be found with bronchoscopy or imaging tests.

In certain embodiments, a therapy disclosed herein may be instituted in addition to surgery to remove a portion of the lung such as a lobectomy, sleeve resection, segmentectomy, or wedge resection.

In certain embodiments, a therapy disclosed herein may be instituted in addition to radiation therapy.

In certain embodiments, the activator of Bak as disclosed herein is administered in combination with an additional chemotherapy agent.

In certain embodiments, the chemotherapy agent is a Bcl-2 inhibitor such as venetoclax, navitoclax, obatoclax, or sabutoclax.

In certain embodiments, the chemotherapy agent is cisplatin, carboplatin, paclitaxel, albumin-bound paclitaxel, docetaxel, gemcitabine, vinorelbine, etoposide, pemetrexed, or combinations thereof.

In certain embodiments, the chemotherapy agent is a combination of cisplatin or carboplatin plus gemcitabine with vinorelbine or paclitaxel.

In certain embodiments, this disclosure relates to a method of treating leukemia comprising administering an effective amount of a Bak activator such as (BKA-073) 1-((2-((2-methoxyacridin-9-yl)amino)ethyl)amino)propan-2-ol, derivative, prodrug, or salt thereof in combination with a venetoclax or other Bcl-2 inhibitor to a subject in need thereof.

In certain embodiments, this disclosure relates to a method of treating leukemia comprising administering an effective amount of a Bak activator such as (BKA-073) 1-((2-((2-methoxyacridin-9-yl)amino)ethyl)amino)propan-2-ol, derivative, prodrug, or salt thereof in combination with a rituximab and venetoclax or other Bcl-2 inhibitor to a subject in need thereof. In certain embodiments, the subject is diagnosed with chronic lymphocytic leukemia (CLL) or relapsed or refractory chronic lymphocytic leukemia (CLL). In certain embodiments, the subject is diagnosed with acute myeloid leukemia (AML) for a treatment using a Bak activator such as (BKA-073) 1-((2-((2-methoxyacridin-9-yl)amino)ethyl)amino)propan-2-ol, derivative, prodrug, or salt thereof in combination with hypomethylating agents, such as decitabine and azacitidine, or cytarabine.

In certain embodiments, a cancer therapy disclosed herein may be instituted in a subject diagnosed with a gene mutation, such as a mutation in Akt, Mcl-1, EGFR, ALK, ROS1, BRAF, RET, MET, NTRK genes or combinations thereof.

In certain embodiments, the subject is diagnosed with an Akt gene mutation (e.g., L52R, Q79K, and D323H). In certain embodiments, the subject is administered a Bak activator disclosed herein in combination with an Akt inhibitor such as capivasertib and ipatasertib. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with an Mcl-1 gene mutation. In certain embodiments, the subject is administered a Bak activator disclosed herein in combination with an Mcl-1 inhibitor disclosed herein. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with an ALK gene mutation. In certain embodiments, the subject is administered a Bak activator disclosed herein in combination with a ALK inhibitor. In certain embodiments, the Bak activator is (BKA-073) 1-((2-((2-methoxyacridin-9-yl)amino)ethyl)amino)propan-2-ol, derivative, prodrug, or salt thereof. In certain embodiments, the subject is administered Bak activator disclosed herein in combination with crizotinib, alectinib, brigatinib, lorlatinib, foretinib, alvotinib, belizatinib, repotrectinib, entrectinib, or ensartinib.

In certain embodiments, the subject is diagnosed with an EGFR gene mutation. In certain embodiments, the subject is administered a Bak activator disclosed herein in combination with an EGFR inhibitor. In certain embodiments, the Bak activator is (BKA-073) 1-((2-((2-methoxyacridin-9-yl)amino)ethyl)amino)propan-2-ol, derivative, prodrug, or salt thereof. In certain embodiments, the subject is administered a Bak activator disclosed herein in combination with afatinib, erlotinib, or lapatinib.

In certain embodiments, the subject is diagnosed with a ROS1 gene mutation. In certain embodiments, the subject is administered a Bak activator disclosed herein in combination with crizotinib, entrectinib, or ceritinib. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with a BRAF gene mutation. In certain embodiments, the subject is administered a Bak activator disclosed herein in combination with dabrafenib or trametinib. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with a RET gene mutation. In certain embodiments, the subject is administered a Bak activator disclosed herein in combination with selpercatinib or pralsetinib. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with a MET gene mutation. In certain embodiments, the subject is administered a Bak activator disclosed herein in combination with capmatinib. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with a NTRK gene mutation. In certain embodiments, the subject is administered a Bak activator disclosed herein in combination with larotrectinib or entrectinib. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with tumors or cancer cells with higher than normal levels of PD-L1. In certain embodiments, the subject is administered a Bak activator disclosed herein in combination with PD-L1 antibody, pembrolizumab, atezolizumab, nivolumab, or ipilimumab. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is administered a Bak activator disclosed herein in combination with bevacizumab for treating cancer. In certain embodiments, the Bak activator is (BKA-073) 1-((2-((2-methoxyacridin-9-yl)amino)ethyl)amino)propan-2-ol, derivative, prodrug, or salt thereof.

In certain embodiments, the subject is diagnosed with squamous cell NSCLC. In certain embodiments, the subject is administered a Bak activator disclosed herein in combination with necitumumab. In certain embodiments, the Bak activator is (BKA-073) 1-((2-((2-methoxyacridin-9-yl)amino)ethyl)amino)propan-2-ol, derivative, prodrug, or salt thereof.

### Bak Activators for Cancer Therapy

Bak is a pro-apoptotic protein required for programmed cell death and apoptosis induction in cancer cells. Experiments reported herein indicate that elevated Bak expression is correlated with poor prognosis in lung cancer, indicating that Bak is a promising prognostic indicator and a potential therapeutic target in lung cancer patients. Here, BKA-073 was identified as a Bak activator, that targets the BH3 domain of Bak, activates the pro-apoptotic function of Bak, and exhibits potent antitumor activity against lung and other cancers. Experiments indicate that BKA-073 directly binds to Bak protein and induces Bak oligomerization in mitochondria leading to activation of a proapoptotic function. Experiments indicate that BKA-073-induced Bak oligomerization promotes mitochondrial priming and Cyt c release, which are early changes in net pro-apoptotic signaling at the mitochondria. BKA-073-induced mitochondrial priming and Cyt c release leads to apoptotic cell death of lung cancer cells. Knockout of Bak but not Bax results in BKA-073 resistance in lung cancer cells and in lung cancer xenografts, indicating that the anti-tumor activity of BKA-073 occurs in a Bak-dependent manner. Furthermore, exogenous expression of wild-type, but not the deleted BH3 mutant, Bak in A549 Bak double negative cells can restore sensitivity to BKA-073, indicating that the apoptotic effect of BKA-073 involve binding to the BH3 domain in the Bak protein. Although it is not intended that embodiments of this disclosure be limited by any particular mechanism, the experimental results indicate a mechanistic model of using a small-molecule Bak activator for cancer therapy.

Several Bax/Bak-independent mechanisms of apoptotic cell death have been described. Experiment reported herein indicate, a small percentage of apoptotic cell death (about 20%) was observed in the Bak−/− and DKO A549 cells. It is contemplated that, in addition to the major Bak-dependent apoptosis mechanism, BKA-073 induces a small proportion of apoptotic cell death (about 20%) through Bax/Bak-independent mechanism(s).

BKA-073 exhibited potent antitumor activity against lung cancer via induction of Bak activation (oligomerization) and apoptotic cell death in xenografts derived from either a lung cancer cell line or a patient-derived SCLC tumor. A dose range between 5 and 15 mg/kg/day was effective without weight loss or significant organ toxicities. As BKA-073 suppressed the growth of patient-derived xenograft (PDX) raised from two patients with refractory SCLC, BKA-073 appears to have clinical utility for human patients.

### Screening of Small Molecules Targeting the BH3 Binding Pocket of Bak

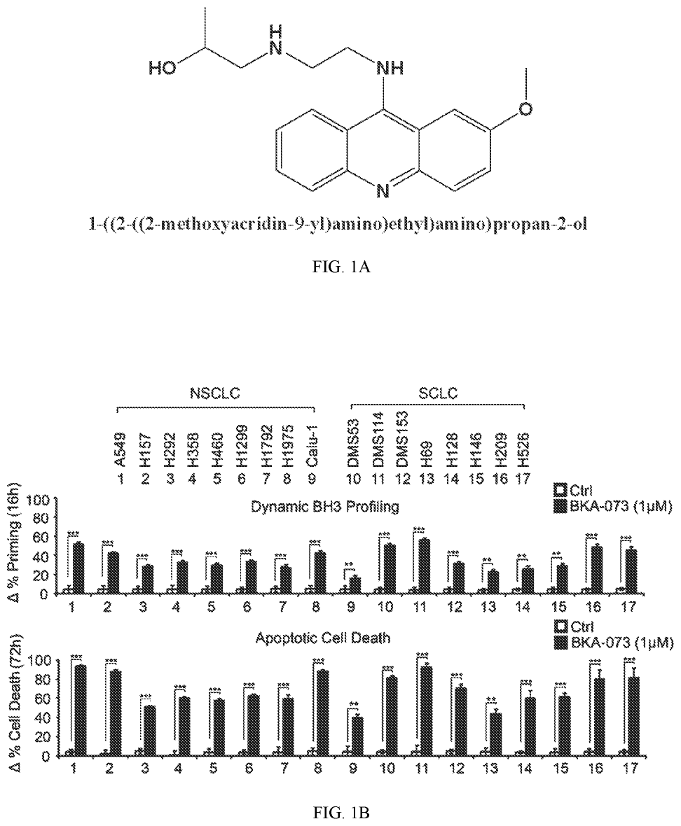

The BH3 death domain is required for the proapoptotic function of Bak. The BH3 domain binding pocket (aa75-88) of Bak (PDB ID: 2YV6) was chosen as a docking site for screening of small molecules using the UCSF DOCK 6.1 program suite and the NCI chemical library (300,000 small molecule) database. Small molecules were ranked according to their energy scores. The top 500 compounds determined to have the highest affinity for the BH3 domain were obtained from the NCI and tested for cytotoxicity in human lung cancer cells (H1299, H460 and A549 cells) by sulforhodamine B (SRB) assay for further screening. Among these small molecules, the compound NSC14073 had the most potent activity against human lung cancer cells. This Bak activator compound was named BKA-073 (C19H24C1N3O2, MW: 361.87) (FIG. 1A).

To test the effect of BKA-073 on mitochondrial priming (Δ % priming) and apoptotic cell death, human lung cancer A549 cells were treated with increasing concentrations (0, 0.25, 0.5, 0.75, 1.0 μM) of BKA-073, followed by analysis of dynamic BH3 profiling (DBP) at 16 h and apoptotic cell death at 72 h. DBP is a functional assay that can measure early changes in net pro-apoptotic signaling at the mitochondrion (“priming”) induced by chemotherapeutic agents or targeted agents in cancer cells. Priming is a measure of how close a cell is to the threshold of apoptosis. Results indicated that BKA-073 induced mitochondrial priming and apoptosis in a dose-dependent manner.

A panel of NSCLC and SCLC cell lines were tested. BKA-073 potently induced mitochondrial priming and apoptosis in both NSCLC and SCLC cell lines that express various levels of endogenous Bak (FIG. 1). NSCLC cell lines (A549, H157 and H1975) and SCLC cell lines (i.e. DMS53, DMS114, H209 and H526) that express relatively higher levels of Bak were more sensitive to BKA-073. In contrast, lung cancer cell lines expressing relatively lower levels of endogenous Bak (NSCLC cell line: Calu-1; SCLC cell lines: H69, H128 and H146) were less sensitive to BKA-073. Thus, the sensitivity of BKA-073 induction of mitochondrial priming and apoptosis is relatively dependent on Bak expression level. In addition to lung cancer cell lines, the efficacy of BKA-073 was evaluated in other types of cancer cell lines, including breast cancer (MDA-MB-231 and MCF7), colon cancer (HCT-116), lymphoma (Ramos), multiple myeloma (OPM-1), pancreatic cancer (PANC-1) and osteosarcoma (U2OS) cell lines. Results revealed that BKA-073 also potently induced mitochondrial priming and apoptotic cell death in various types of cancer cell lines (FIG. 1C), suggesting that BKA-073 should be effective in various cancer types.

### BKA-073 Directly Binds to Bak Protein and Induces Bak Oligomerization Leading to Cyt c Release

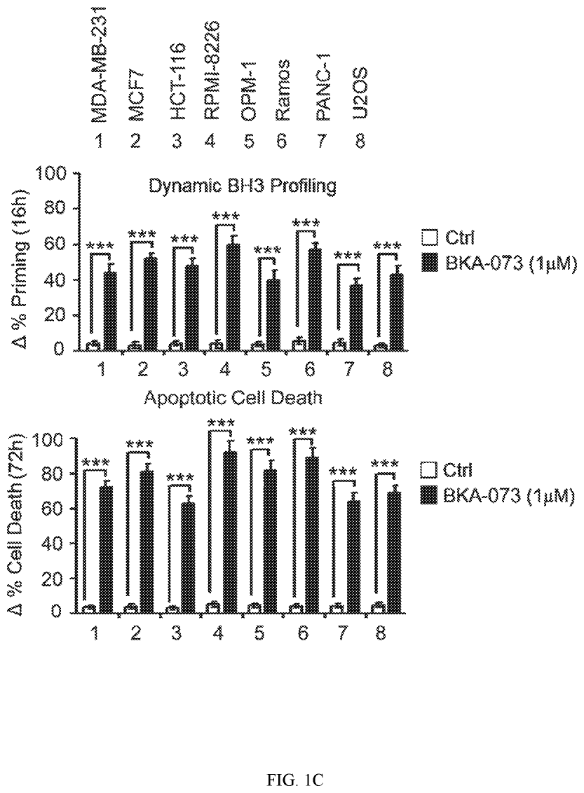

To confirm the binding of BKA-073 with Bak, a competitive fluorescence polarization (FP) assay was conducted using purified human Bak protein, fluorescent Bak BH3 domain peptide, and BKA-073. BKA-073 directly bound to human Bak protein with high binding affinity (Ki: 72.3±5.96 nM). Specifically, BKA-073 had very low binding affinity with other Bcl2 family members (FIG. 2A), indicating that it selectively binds to Bak. There are multiple amino acid differences in the BH3 domains between Bak and other Bcl2 family members. BKA-073 appears to only binds to Bak but not to other Bcl2 family members (Bax, Bcl2, Bcl-XL, Bcl-w and Mcl-1).

Isothermal titration calorimetry (ITC) was also employed to measure Bak/BKA-073 binding. ITC is a direct, label- and immobilization-free technique which measures the binding affinity between proteins and small molecule ligands that interact with each other, and it can be used to analyze binding constant (Kd) values in the millimolar and nanomolar range. ITC experiments were performed to assess BKA-073/Bak binding using an auto-iTC200 instrument. Results indicate that BKA-073 directly bound human Bak protein with nanomolar range binding affinity (Kd=88.62±5.73 nM) (FIG. 2B). In contrast, BKA-073 failed to bind to the BH3 deletion human Bak mutant protein (ΔBH3) in the ITC assay suggesting that the BH3 domain is involved for Bak to interact with BKA-073.

In addition to human Bak/BKA-073 binding, mouse Bak/BKA-073 binding was measured using ITC. BKA-073 also directly bound mouse Bak protein with good binding affinity (Kd=93.37±7.91 nM). These experiments indicate that BKA-073 can bind both human and mouse Bak proteins.

A step in the apoptosis process is the oligomerization of Bak. To assess whether BKA-073 affects the ability of Bak to form oligomers in the mitochondrial membrane, a cross-linking study with Bis (maleimido) hexane (BMH) was carried out. Treatment of A549 cells with BKA-073 (1 μM) facilitated the formation of Bak dimers and trimers. The molecular sizes of these adducts were estimated to be multiples of about 28 kDa, suggesting the formation of Bak homo-oligomers in A549 cells. These findings indicate that BKA-073 can activate Bak through its oligomerization in mitochondria. Formation of Bak oligomers in mitochondria promotes cytochrome c (Cyt c) release to induce apoptosis. Experiments indicate that BKA-073-induced Bak oligomerization promoted Cyt c release from mitochondria in A549 cells.

### BKA-073 Potently Suppresses NSCLC Xenografts Via Induction of Apoptosis in a Bak-Dependent Manner

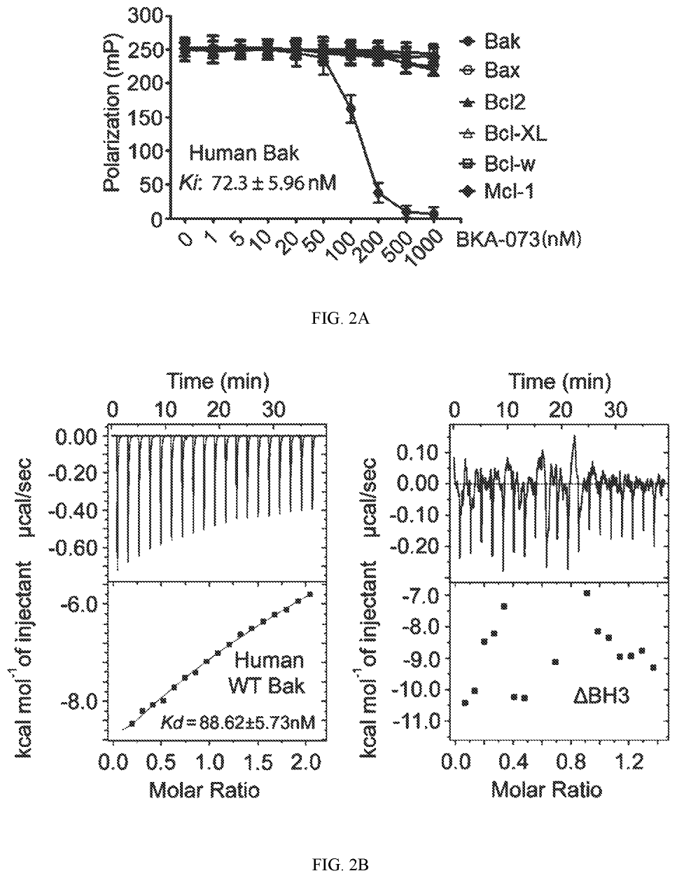

To test the potency of BKA-073 in vivo, mice carrying lung cancer xenografts derived from A549 cells were treated i.p. with increasing doses (0, 5, 10, 15 mg/kg/d) of BKA-073 for 28 days. BKA-073 potently suppressed lung cancer growth in a dose-dependent fashion (FIG. 3). To assess whether BKA-073 induced suppression of tumor growth occurs through activation of Bak and apoptosis in vivo, representative samples from harvested tumor tissues were analyzed by cross-linking with BMH for Bak oligomerization or by immunohistochemistry (IHC) for active caspase 3. Dose-dependent Bak oligomerization and apoptosis were observed in tumor tissues after BKA-073 treatment. Importantly, doses of 5 to 15 mg/kg/d not only potently suppressed tumor growth but were also well tolerated without significant toxicity to mice. Doses between 5 mg/kg and 15 mg/kg provide an optimal therapeutic index for BKA-073 for in vivo experimentation involving lung cancer xenografts.

### BKA-073 Exhibits Potent Antitumor Activity Against SCLC in Xenografts and PDX Models

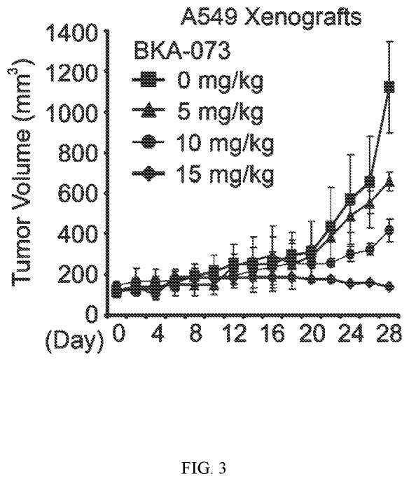

To further evaluate the anti-tumor activity of BKA-073 against SCLC in vivo, mice carrying SCLC xenografts derived from the DMS114 cell line or patient-derived xenografts (PDXs) from two patients with refractory SCLC (TKO-2 and TKO-5) were treated i.p. with BKA-073 (15 mg/kg/d) for 2-4 weeks. BKA-073 potently suppressed tumor growth of DMS114 xenografts and SCLC PDXs, which occurred through induction of apoptosis (FIG. 4). These findings indicate that BKA-073 may potentially be effective in patients with SCLC.

### BKA-073 Represses Mutant KRAS-Driven Lung Cancer Growth and Prolongs Survival in Genetically Engineered Mouse Models (GEMMs)

KRAS is a commonly mutated oncogene, yet no effective targeted therapies exist for KRAS-mutant cancers. Intriguingly, expression of exogenous constitutively active KRAS (G12D) mutant in H1944 cells with wild-type KRAS background significantly enhanced Bak expression. Since BKA-073 is able to induce apoptosis by activation of Bak via facilitating its oligomerization in vitro and in vivo, experiments were performed to determine whether BKA-073 is effective for the treatment of mutant KRAS-driven cancer.

To assess the potency of BKA-073 in KRAS mutant driven lung cancer, lox-stop-lox (LSL)-KRAS G12D LKB1fl/fl (i.e. KL) mice were generated and bred. These mice contain a KRAS G12D LSL knock-in allele and a floxed allele of LKB1 (LKB1fl/fl). Primary lung adenocarcinoma was detectable as early as 6 weeks after intranasal administration of 5×10⁶ pfu adenovirus expressing Cre recombinase (AdeCre) in KRAS G12D LKB1fl/fl (KL) mice. Increased Bak expression was observed in tumor tissues from KL mice as compared to adjacent normal lung tissues of representative cross-sections of each lung lobe for each mouse. Results indicated that treatment of KL mice with BKA-073 resulted in significant reduction of tumor burden and multiplicity in the lung via apoptosis. Treatment with BKA-073 significantly prolonged survival of KL mice, thereby providing a strong rationale to employ Bak agonist BKA-073 for the treatment of mutant KRAS-driven lung cancer.

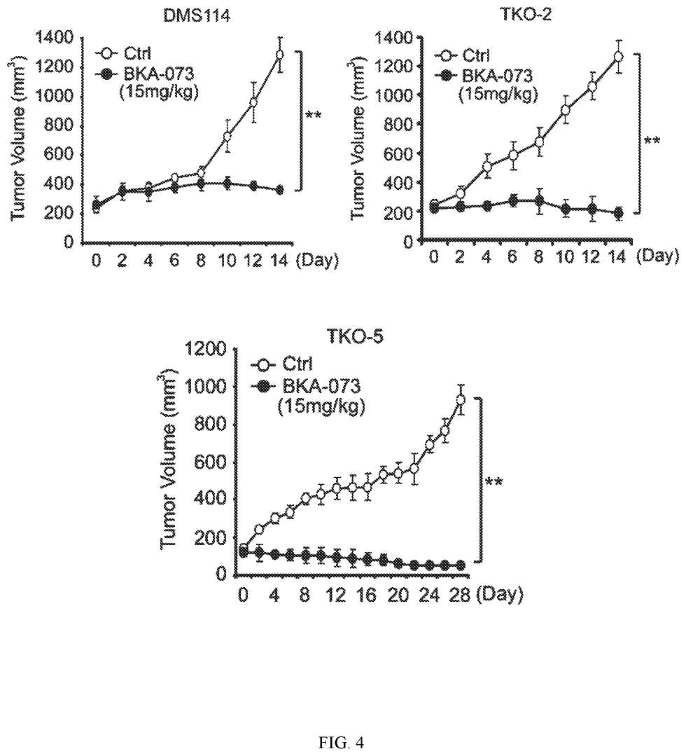

To further assess the potential of BKA-073 as therapy for mutant KRAS-driven lung cancer, BKA-073 (15 mg/kg/d) or vehicle was administered to KL mice i.p. starting at 6 weeks post AdeCre delivery. After treatment for 48 days, KL mice were euthanized with carbon dioxide asphyxiation. Lungs with tumor and normal lung tissues were collected for further analysis. To quantify tumor burden and tumor multiplicity in mice, H&E-stained lungs were imaged with morphometric software to quantify the surface area composed of tumor as opposed to normal tissue compared with the control group. There were 4 deaths out of 6 mice in the control group versus 2 deaths out of 6 mice in the BKA-073 treatment group (p<0.01), calculated up to 48 days before euthanization (FIG. 5).

### Bak Accumulates in Radioresistant Lung Cancer Cells and BKA-073 Reverses Radioresistance In Vitro and In Vivo

To further investigate whether Bak contributes to radioresistance, three lung cancer cell lines were established with ionizing radiation resistance (i.e. A549-IRR, H358-IRR and H460-IRR). Increased levels of Bak were observed in A549-IRR, H358-IRR and H460-IRR cells as compared to parental A549 (A549-P), H358 (H358-P) and H460 (H460-P) cells. A549-IRR, H358-IRR and H460-IRR cells grow well under cell culture conditions, indicating that Bak molecules are in an inactive form under normal growth conditions. A549-P, H385-P and H460-P cells remained sensitive to IR, but A549-IRR, H358-IRR and H460-IRR became insensitive to IR. Both parental and radioresistant cell lines were sensitive to BKA-073, suggesting that BKA-073 is efficacious in radioresistant cells.

To further test this in vivo, NSCLC xenografts derived from A549-P and A549-IRR cell lines were treated with IR (2Gy/exposure, every other day for total of 5 times) or BKA-073 (15 mg/kg/d) for 4 weeks. Lung cancer xenografts derived from A549-IRR cells were resistant to IR treatment whereas xenografts derived from A549-P were sensitive to IR treatment. BKA-073 repressed xenografts derived from either A549-P or A549-IRR cells, indicating that BKA-073 is also efficacious in radioresistant lung cancer xenografts.

Combination of BKA-073 with Bcl2 Inhibitor Venetoclax (ABT-199) Synergistically Suppresses Lung Cancer In Vitro and In Vivo

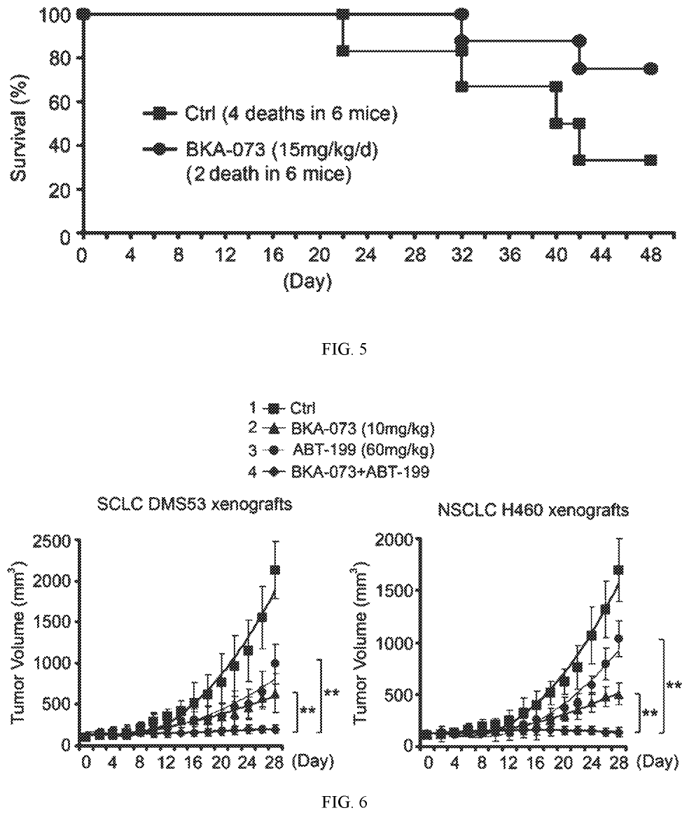

To test whether direct activation of the proapoptotic activity of Bak combined with inhibition of the antiapoptotic function of Bcl2 achieves synergistic effects in lung cancer therapy, a SCLC cell line (DMS53) and NSCLC cell line (H460) that express endogenous Bcl2 and Bak were treated with venetoclax in combination with BKA-073 for 16 h and 72 h, followed by analysis of dynamic BH3 profiling and apoptosis, respectively. BKA-073 in combination with venetoclax exhibited strong synergism in the induction of mitochondrial priming and apoptosis in both SCLC and NSCLC lines. SCLC xenografts derived from DMS53 cells and NSCLC xenografts derived from H460 cells were treated with BKA-073 (10 mg/kg/d) i.p., venetoclax (60 mg/kg/d) orally, or the combination for 4 weeks. Results revealed that combined treatment with BKA-073 and venetoclax synergistically suppressed both SCLC and NSCLC in vivo (FIG. 6).

Higher Levels of Bak in Tumor Tissues are Correlated with Poor Prognosis of Patients with NSCLC

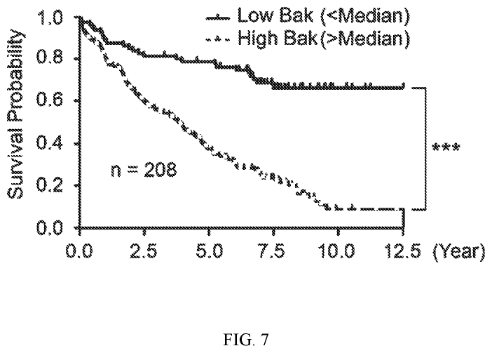

Higher levels of endogenous Bak expression were observed in various human lung cancer cell lines, which did not cause apoptosis in cell culture medium without any treatment. This indicates that Bak protein is an inactive form under normal growth conditions. To further test whether Bak is upregulated in tumor tissues from NSCLC patients, Bak expression was analyzed in samples from 208 NSCLC patients by IHC staining using Bak antibody. Formalin-fixed and paraffin-embedded human tissue samples were obtained. Tissue microarray (TMA) was constructed with replicate cores of tumor and adjacent normal lung. The semiquantitative evaluation of IHC staining of Bak was carried out using an “immunoscore” based on both the percentage of stained cells and staining intensity as described. Bak protein expression was significantly higher in tumor tissues compared to adjacent normal lung tissues. Importantly, increased Bak expression in tumor tissues were correlated with poor prognosis of NSCLC patients (FIG. 7) indicating that Bak is a potential prognostic biomarker for NSCLC. These experiments indicate that the Bak activator (BKA-073) is an effective strategy to improve the outcome of NSCLC patients and other cancers.

1. A method of treating cancer comprising administering an effective amount of a Bak activator to a human subject in need thereof.

2. The method of claim 1 wherein the Bak activator is 1-((2-((2-methoxyacridin-9-yl)amino)ethyl)amino)propan-2-ol (BKA-073), derivative, ester, or salt thereof.

3. The method of claim 1 wherein the Bak activator is a compound of formula I or II, 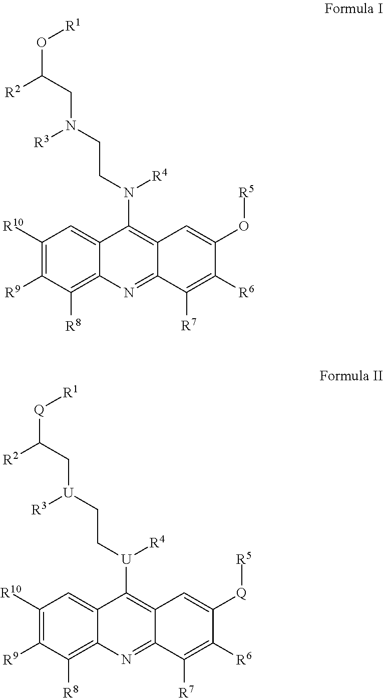
ester, or salts thereof, wherein:
Q is O or S;
U is N or CH;
R¹, R², R³, R⁴, R⁵, R⁶, R⁷, R⁸, R⁹, and R¹⁰ are each individually and independently hydrogen, alkyl, halogen, cyano, hydroxy, amino, mercapto, formyl, carboxy, carbamoyl, alkoxy, alkanoyl, alkylthio, alkylamino, aminoalkyl, (alkyl)₂amino, phosphate, alkylsulfinyl, alkylsulfonyl, arylsulfonyl, carbocyclyl, aryl, or heterocyclyl, wherein R¹, R², R³, R⁴, R⁵, R⁶, R⁷, R⁸, R⁹, and R¹⁰ are optionally substituted with one or more, the same or different, R¹¹;
R¹¹ is alkyl, halogen, cyano, hydroxy, amino, mercapto, formyl, carboxy, carbamoyl, alkoxy, alkanoyl, alkylthio, alkylamino, phosphate, aminoalkyl, (alkyl)₂amino, alkylsulfinyl, alkylsulfonyl, arylsulfonyl, carbocyclyl, aryl, or heterocyclyl, wherein R¹¹ is optionally substituted with one or more, the same or different, R¹²;
R¹² is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, 2-methoxyethoxy, 2-hydroxyethoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

4. The method of claim 3 wherein R¹ is hydrogen.

5. The method of claim 3 wherein R² is alkyl.

6. The method of claim 3 wherein R³ is hydrogen.

7. The method of claim 3 wherein R⁴ is hydrogen.

8. The method of claim 3 wherein R⁵ is alkyl.

9. The method of claim 3 wherein R⁶, R⁷, R⁸, R⁹, and R¹⁰ are hydrogen.

10. The method of claim 3 wherein Q is O.

11. The method of claim 3 wherein U is NH.

12. The method of claim 1, wherein subject is a human.

13. The method of claim 1 wherein the subject is diagnosed with non-small cell lung cancer.

14. The method of claim 1, wherein the Bak activator is administered in combination with an additional chemotherapy agent.

15. The method of claim 14, wherein the chemotherapy agent is a Bcl-2 inhibitor.

16. The method of claim 15 wherein the Bcl-2 inhibitor is venetoclax, navitoclax, obatoclax, sabutoclax.

17. A pharmaceutical composition comprising a Bak activator disclosed herein or pharmaceutically acceptable salt thereof and a pharmaceutically acceptable excipient.

18. The pharmaceutical composition of claim 17 in the form of a pill, capsule, or table.

19. The pharmaceutical composition of claim 17 in the form of an aqueous isotonic or non-isotonic pH buffered solution.

20. The pharmaceutical composition of claim 17, wherein the pharmaceutically acceptable excipient is selected from a saccharide, disaccharide, sucrose, lactose, glucose, mannitol, sorbitol, polysaccharides, starch, cellulose, microcrystalline cellulose, cellulose ether, hydroxypropyl cellulose (HPC), xylitol, maltitol, gelatin, polyvinylpyrrolidone (PVP), polyethylene glycol (PEG), hydroxypropyl methylcellulose (HPMC), crosslinked sodium carboxymethyl cellulose, dibasic calcium phosphate, calcium carbonate, stearic acid, magnesium stearate, talc, magnesium carbonate, silica, vitamin A, vitamin E, vitamin C, retinyl palmitate, selenium, cysteine, methionine, citric acid, and sodium citrate, methyl paraben, propyl paraben, and combinations thereof.
